(19) United States
(12) Patent Application Publication
(10) Pub. No.: US 2024/0382302 A1
(43) Pub. Date: Nov. 21, 2024

(54) ESOPHAGEAL SLEEVE DEVICES AND METHODS OF MAKING THE SAME

(71) Applicants: Georgia Tech Research Corporation, Atlanta, GA (US); The Johns Hopkins University, Baltimore, MD (US)

(72) Inventors: Scott J. Hollister, VIII, Atlanta, GA (US); Shaun Kunisaki, Ann Arbor, MI (US); Sriharsha Ramaraju, Atlanta, GA (US)

(21) Appl. No.: 18/288,252

(22) PCT Filed: May 19, 2022
(86) PCT No.: PCT/US2022/072445

§ 371 Date: Oct. 25, 2023

Related U.S. Application Data
(60) Provisional application No. 63/190,434, filed on May 19, 2021.
(60) Provisional application No. 63/272,206, filed on Oct. 27, 2021.

(51) Int. Cl. A61F 2/04; A61L 27/18; A61L 27/54; A61L 27/58

(52) U.S. Cl. CPC A61F 2/04; A61L 27/18; A61L 27/54; A61L 27/58; A61F 2002/044; A61F 2210/0004; A61F 2210/0023; A61F 2210/0071; A61F 2240/004; A61L 2400/16; A61L 2430/22

(57) ABSTRACT

Disclosed herein are esophageal sleeve devices comprising bioresorbable scaffolds having a first shape and a second shape. The bioresorbable scaffolds can be made from a shape memory polymer comprising at least one monomer unit of glycerol and at least one monomer unit of dodecanedioate. The bioresorbable scaffolds can also have a functionalized surface modified to have a biology corresponding to a patient. The bioresorbable scaffolds can take the first shape at a first environmental temperature and the second shape at a second environmental temperature. the second environmental temperature being greater than the first environmental temperature. Also disclosed herein are methods of implanting the same.

### CROSS-REFERENCE TO RELATED APPLICATIONS

This application claims the benefit of U.S. Provisional Application Ser. No. 63/272,206, filed on 27 Oct. 2021, and U.S. Provisional Application Ser. No. 63/190,434, filed on 19 May 2021, the entire contents and substance of which are incorporated herein by reference in their entirety as if fully set forth below.

### FIELD OF THE DISCLOSURE

The present disclosure relates generally to esophageal sleeve devices and methods. Particularly, embodiments of the present disclosure relate to esophageal sleeve devices made from bioresorbable implant materials comprising shape memory polymers and methods of making the same.

### BACKGROUND

Congenital esophageal atresia (EA), with or without tracheoesophageal fistula, is a relatively common birth defect of unknown etiology resulting in a complete discontinuity of the esophagus. Although neonatal primary surgical repair permanently restores esophageal continuity in short-gap EA (<3 cm between proximal and distal segments), the procedure is fraught with a high rate of postoperative complications, including leaks (23%), recurrent strictures (43%), and recurrent fistulae (5%), resulting in the need for additional procedures and prolonged hospitalization. Children also suffer long-term complications despite successful repair, including gastroesophageal reflux (95%) and chronic dysphagia. A major contributor to the high postoperative complication rate and poor long-term function of the esophagus after neonatal EA repair is the high longitudinal stress placed on the delicate and ischemic anastomosis. During the procedure, the upper and lower ends of the esophagus are usually separated by a 2-4 cm gap. In order to bring the two ends together, the ends need to be dissected away from adjacent posterior mediastinal structures and are devascularized in the process. Increased longitudinal tension in combination with a poor blood supply increase the risk of anastomotic complications and impair long-term function after esophageal repair.

Reducing complications from esophageal repair therefore requires strategies that reduce longitudinal tension at the anastomosis while also improving blood supply. Primary repair of the esophagus can be modified to reduced anastomotic tension, however, this strategy alone does not allow for the possibility to improve blood supply.

Although various animal models of esophageal mechanics have been previously described, detailed understanding of esophageal atresia repair mechanics describing stress distribution at the anastomosis arising from primary repair is limited. Furthermore, design requirements for biomaterials or devices to reduce anastomotic tension and generally suitable for esophageal repair are also limited. Evaluating mechanical properties of the esophagus and retraction forces from anastomotic tension provides a baseline for modeling and therefore better understanding of anastomotic repair. The esophagus is comprised of a muscularis externa layer, a mucosal layer and a submucosal layer. A combination of equibiaxial and inflation testing were conducted on porcine esophagi to approximate mechanics of human esophageal tissues and generate 3D models. These multiaxial tests are fit to strain energy functions with exponential, polynomial or a hybrid constitutive models describing the deformation behavior. Additionally, each layer of esophageal tissue is fit to a strain energy function describing the mechanical behavior. Idealized computational models of esophageal atresia that incorporate these nonlinear esophageal properties and apply them to relevant patient geometries and gap lengths can provide insights for reducing anastomotic tension using primary repair or scaffolding materials.

Specifically, in the context of esophageal atresia repair for short gap and long gap scenarios, modeling anastomotic tension arising from inherent retraction through the use of body forces can provide critical insight towards optimizing repair strategies. Prior experimental studies in human cadaveric tissues and pigs investigated the relationship between gap length, body force and the safety limits for retraction forces with respect to stricture formation. Although no devices have been used explicitly towards reinforcing the anastomosis at the time of repair, various overlays and underlays are used to reduce anastomotic tension and heal the defect. Additionally, the bulk of esophageal tissue repair applications to date address segmental defect reconstruction utilizing natural ECM and synthetic degradable and non-degradable polymers. Limitations using these materials suggest the need for controlling degradation rates while providing appropriate mechanical support throughout the healing timeframe. Natural extracellular matricies undergo rapid resorption and degradation resulting in recurrence whereas synthetic polymers lack the inherent mechanical properties for repair. However, synthetic polymers can more readily be tuned to exhibit a range of mechanical properties through control of molecular weight, co-polymerization with softer polymers or gels, or creation of composites. For instance, poly(L-lactide-co-caprolactone) PLCL is a copolymer of lactide and caprolactone exhibiting softer mechanical properties than either poly lactic acid or poly caprolactone alone.

What is needed, therefore, are esophageal sleeve devices and methods of making the same. Embodiments of the present disclosure address this need as well as other needs that will become apparent upon reading the description below in conjunction with the drawings.

### BRIEF SUMMARY OF THE DISCLOSURE

The present disclosure relates generally to esophageal sleeve devices and methods. Particularly, embodiments of the present disclosure relate to esophageal sleeve devices made from bioresorbable implant materials comprising shape memory polymers and methods of making the same.

An exemplary embodiment of the present disclosure can provide an esophageal sleeve device comprising: a bioresorbable scaffold having a first shape and a second shape, the bioresorbable scaffold comprising: a shape memory polymer comprising at least one monomer unit of glycerol and at least one monomer unit of dodecanedioate; and a functionalized surface modified to have a biology corresponding to a patient, wherein the bioresorbable scaffold takes the first shape at a first environmental temperature and the second shape at a second environmental temperature, the second environmental temperature being greater than the first environmental temperature.

In any of the embodiments disclosed herein, the esophageal sleeve can be delivered when the bioresorbable scaffold is in the second shape at the second environmental temperature, and the esophageal sleeve can be implanted in the first shape at the first temperature.

In any of the embodiments disclosed herein, the shape memory polymer can have a melt transition temperature from approximately 25° C. to approximately 45° C., and the melt transition temperature can be greater than or equal to the first environmental temperature and less than the second environmental temperature.

In any of the embodiments disclosed herein, the melt transition temperature can be from approximately 31° C. to 35° C.

In any of the embodiments disclosed herein, the shape memory polymer can be an elastomer above the melt transition temperature a thermoplastic in the below the melt transition temperature.

In any of the embodiments disclosed herein, the first shape can be a curved or tubular shape.

In any of the embodiments disclosed herein, the second shape can be a tubular shape comprising a cut along a longitudinal axis of the bioresorbable scaffold.

In any of the embodiments disclosed herein, the functionalized surface can comprise a plurality of suture holes cut into the functionalized surface.

In any of the embodiments disclosed herein, the functionalized surface can comprise at least one functional group bonded to the shape memory polymer.

In any of the embodiments disclosed herein, the at least one functional group can comprise a bioactive agent.

In any of the embodiments disclosed herein, the molar ratio of the at least one monomer unit of glycerol to the at least one monomer unit of dodecanedioate can be from approximately 10:1 to approximately 1:10.

In any of the embodiments disclosed herein, the bioresorbable scaffold can have a biodegradation time when implanted in vivo from approximately 2 months to approximately 24 months.

Another embodiment of the present disclosure can provide a method of implanting an esophageal sleeve, the method comprising: delivering the esophageal sleeve to a patient, the esophageal sleeve comprising a bioresorbable scaffold having a first shape and a second shape, wherein the bioresorbable sleeve is in the second shape during the delivering; recovering the esophageal sleeve such that the bioresorbable sleeve takes the first shape when implanted in the patient.

In any of the embodiments disclosed herein, the bioresorbable scaffold can take the first shape at a first environmental temperature and the second shape at a second environmental temperature, the second environmental temperature being greater than the first environmental temperature.

In any of the embodiments disclosed herein, the bioresorbable scaffold can comprise a shape memory polymer comprising at least one monomer unit of glycerol and at least one monomer unit of dodecanedioate; and a functionalized surface.

In any of the embodiments disclosed herein, the shape memory polymer can have a melt transition temperature from approximately 25° C. to approximately 45° C., and the melt transition temperature can be greater than or equal to the first environmental temperature and less than the second environmental temperature.

In any of the embodiments disclosed herein, the melt transition temperature can be from approximately 31° C. to 35° C.

In any of the embodiments disclosed herein, the shape memory polymer can be an elastomer above the melt transition temperature a thermoplastic in the below the melt transition temperature.

In any of the embodiments disclosed herein, the first shape can be a curved or tubular shape.

In any of the embodiments disclosed herein, the second shape can be a tubular shape comprising a cut along a longitudinal axis of the bioresorbable scaffold.

In any of the embodiments disclosed herein, the functionalized surface can comprise a plurality of suture holes cut into the functionalized surface.

In any of the embodiments disclosed herein, the functionalized surface can comprise at least one functional group bonded to the shape memory polymer.

In any of the embodiments disclosed herein, the at least one functional group can comprise a bioactive agent.

In any of the embodiments disclosed herein, the molar ratio of the at least one monomer unit of glycerol to the at least one monomer unit of dodecanedioate can be from approximately 10:1 to approximately 1:10.

In any of the embodiments disclosed herein, the bioresorbable scaffold can have a biodegradation time when implanted in vivo from approximately 2 months to approximately 24 months.

These and other aspects of the present disclosure are described in the Detailed Description below and the accompanying figures. Other aspects and features of embodiments of the present disclosure will become apparent to those of ordinary skill in the art upon reviewing the following description of specific, exemplary embodiments of the present invention in concert with the figures. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments of the invention discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF THE DRAWINGS

The accompanying drawings, which are incorporated in and constitute a part of this specification, illustrate multiple embodiments of the presently disclosed subject matter and serve to explain the principles of the presently disclosed subject matter. The drawings are not intended to limit the scope of the presently disclosed subject matter in any manner.

FIG. 1 illustrates an implant material having a first shape and a second shape in accordance with the present disclosure.

FIG. 2 illustrates a flowchart of a method of making an implant material in accordance with the present disclosure.

FIG. 3 illustrates a flowchart of another method of making an implant material in accordance with the present disclosure.

FIG. 4 illustrates a flowchart of a method of implanting an esophageal sleeve in accordance with the present disclosure.

FIG. 5 is a chart of a model of retraction force for an esophagus for an esophageal sleeve in accordance with the present disclosure.

FIG. 6 illustrates a split view model of an esophageal sleeve in accordance with the present disclosure.

FIG. 7 illustrates a model of an esophageal sleeve with a functionalized surface in accordance with the present disclosure.

FIG. 8 illustrates a model of LaGrangian strain in an esophageal sleeve in accordance with the present disclosure.

FIGS. 9A and 9B illustrate plots of difference in LaGrangian strain and displacement, respectively, for examples of an esophageal sleeve in accordance with the present disclosure.

FIGS. 10A and 10B illustrate reductions in effective LaGrangian strain for examples of an esophageal sleeve in accordance with the present disclosure.

FIG. 11 illustrates LaGrangrian strain plots for examples of an esophageal sleeve in accordance with the present disclosure.

FIG. 12 illustrates displacement magnitude plots for examples of an esophageal sleeve in accordance with the present disclosure.

FIG. 13 illustrates strain energy density plots for examples of an esophageal sleeve in accordance with the present disclosure.

FIG. 14 illustrates models of impacted strain for examples of an esophageal sleeve comprising various shape memory polymers in accordance with the present disclosure.

### DETAILED DESCRIPTION

Designing a sleeve to reduce anastomotic strain at the time of repair can utilize an understanding how the material properties of the sleeve, geometric design of the sleeve, application of sutures and mechanics of esophageal tissues impact tension at the anastomosis.

Sleeve mechanical properties can impact the strain, displacement, and strain energy density at the anastomosis site. Although a stiffer sleeve can provide greater support and reduces displacement at the anastomosis site, a sleeve that allows compliance suited for esophagus tissues can dissipate more strain energy density. Numerous natural, synthetic, and hybrid materials can be used in esophageal support devices. In all such cases, materials used as sleeves might not attenuate anastomotic tension. Additionally, these materials can be used as segmental defect replacements or reinforcements meeting structural requirements to support normal esophageal function.

The mechanical properties of synthetic materials (GPa) previously considered for esophageal repair can be isotropic elastic with elastic moduli and tensile strength several orders of magnitude higher than esophageal tissues. Moreover, such materials may struggle to support the compliant nonlinear anisotropic properties of native esophageal tissues (MPa). This mismatch in tissue mechanics, combined with degradation byproducts can drive chronic inflammation, stricture formation, and restenosis.

Natural materials can meet the nonlinear anisotropic mechanical requirements for esophageal repair but can undergo rapid degradation and resorption causing structural deficits at the reinforcement site. Various hybrid materials comprised of natural and synthetic mixtures can be used in esophageal repair. Disclosed herein is a shape memory polymer exhibiting nonlinear elastic properties tunable to meet various tissue properties with a degradation rate appropriate for soft tissue regeneration applications. Such materials can be mechanically tested to failure and fit to nonlinear elastic constitutive models.

Disclosed herein is an esophageal anastomotic sleeve supporting multiple supporting mattress suture configurations that can displace longitudinal tension away from the most ischemic ends of the esophageal repair to improve anastomotic healing and decrease complications. Additionally, the contributions of suture bite length, suture method for sleeve application, and sleeve mechanical properties can be investigated with respect to atresia gap length. The present disclosure can further be used to develop a computational framework for evaluating primary and device-assisted repair of esophageal atresia.

As disclosed herein, the mucosal and muscle layers of the esophagus can be modeled as nested concentric cylinders having nonlinear elastic properties derived from equibiaxial testing. Retraction forces on the approximated segments can be modeled using body forces to simulate esophageal gap repair tension forces causing esophagus retraction tension previously investigated in a porcine model of esophagus gap repair. Impact of suture bite length on the effective anastomotic strain, total displacement, and strain energy density can be evaluated. The % reduction in anastomotic strain, total displacement, and strain energy density of the elastomeric sleeves using varying suture patterns can be compared to primary repair. Impact of sleeve application for long gap atresia can be assessed compared to primary repair. Finally, the impact of sleeve mechanical properties on the effective anastomotic strain, total displacement, and strain energy density can be evaluated for different esophageal gaps.

Esophageal atresia is a potentially lethal congenital malformation occurring in 1 in every 4100 live births resulting in discontinuity of the esophagus. Treatment requires approximating the disconnected esophageal segments and suturing the ends to restore continuity. Leaks and strictures are prevalent in primary surgical repair of the esophagus especially in the subset of neonates presenting long gap atresia (<3 cm). Extracellular matrix derived scaffolds and biodegradable polymer scaffolds can be used in preclinical models for use in alleviating esophageal anastomotic tension varying degrees of success. Disclosed herein are biodegradable shape memory materials for use in a number of soft tissue repair applications. Developing repair strategies addressing esophageal atresia can use a framework for approximating tension at the anastomosis.

Also disclosed herein is a computational framework for approximating esophageal anastomotic tension to study the impact of primary and device supported repair. The esophagus can be modeled as an idealized concentric cylinder comprised of mucosal and muscle layers described by nonlinear strain energy functions and a mixed fiber model with a Neo-Hookean base material (FEBIO studio). Sutures can be modeled as nonlinear elastic springs carrying only tension, and shape memory polymers can be modeled as nonlinear elastic materials using one-term Ogden parameters. The impact of suture bite (length of suture from anastomosis), sleeve material properties, sleeve suture strategy, and gap length can be evaluated with respect to anastomotic LaGrangian strain, displacement magnitude, and strain energy density. With increasing gap length, there can be an increase in anastomotic strain, displacement magnitude, and strain energy density. Increasing the suture bite length can decrease strain at the anastomosis.

Application of the sleeve can reduce strain, displacement, and strain energy to a greater extent in longer gap atresia. Increasing the number of sutures to apply the sleeve did not decrease the esophageal strain compared to sleeves with lesser number of sutures. Sleeve material testing can implement an interplay between the nonlinear mechanical properties of the selected materials and their contribution to reducing anastomotic tension. Taken together, the present disclosure provides a unique framework for computational verification of design hypothesis broadly addressing clinical procedure optimization, material design, and device design for surgical repair of esophageal atresia.

Although certain embodiments of the disclosure are explained in detail, it is to be understood that other embodiments are contemplated. Accordingly, it is not intended that the disclosure is limited in its scope to the details of construction and arrangement of components set forth in the following description or illustrated in the drawings. Other embodiments of the disclosure are capable of being practiced or carried out in various ways. Also, in describing the embodiments, specific terminology will be resorted to for the sake of clarity. It is intended that each term contemplates its broadest meaning as understood by those skilled in the art and includes all technical equivalents which operate in a similar manner to accomplish a similar purpose.

Herein, the use of terms such as “having,” “has,” “including,” or “includes” are open-ended and are intended to have the same meaning as terms such as “comprising” or “comprises” and not preclude the presence of other structure, material, or acts. Similarly, though the use of terms such as “can” or “may” are intended to be open-ended and to reflect that structure, material, or acts are not necessary, the failure to use such terms is not intended to reflect that structure, material, or acts are essential. To the extent that structure, material, or acts are presently considered to be essential, they are identified as such.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

It is also to be understood that the mention of one or more method steps does not preclude the presence of additional method steps or intervening method steps between those steps expressly identified.

The components described hereinafter as making up various elements of the disclosure are intended to be illustrative and not restrictive. Many suitable components that would perform the same or similar functions as the components described herein are intended to be embraced within the scope of the disclosure. Such other components not described herein can include, but are not limited to, for example, similar components that are developed after development of the presently disclosed subject matter.

The term “aliphatic” or “aliphatic group,” as used herein, means a straight-chain (i.e., unbranched) or branched, substituted or unsubstituted hydrocarbon chain that is completely saturated or that contains one or more units of unsaturation, or a monocyclic hydrocarbon, bicyclic hydrocarbon, or tricyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-30 aliphatic carbon atoms. In some embodiments, aliphatic groups contain 1-20 aliphatic carbon atoms. In other embodiments, aliphatic groups contain 1-10 aliphatic carbon atoms. In still other embodiments, aliphatic groups contain 1-6 aliphatic carbon atoms, and in yet other embodiments, aliphatic groups contain 1, 2, 3, or 4 aliphatic carbon atoms. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “cycloaliphatic,” as used herein, refers to saturated or partially unsaturated cyclic aliphatic monocyclic, bicyclic, or polycyclic ring systems, as described herein, having from 3 to 14 members, wherein the aliphatic ring system is optionally substituted as defined above and described herein. Cycloaliphatic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, norbornyl, adamantyl, and cyclooctadienyl. In some embodiments, the cycloalkyl has 3-6 carbons. The terms “cycloaliphatic,” may also include aliphatic rings that are fused to one or more aromatic or nonaromatic rings, such as decahydronaphthyl or tetrahydronaphthyl, where the radical or point of attachment is on the aliphatic ring. In some embodiments, a carbocyclic group is bicyclic. In some embodiments, a ‘carbocyclic group is tricyclic. In some embodiments, a carbocyclic group is polycyclic. In some embodiments, “cycloaliphatic” (or “carbocycle” or “cycloalkyl”) refers to a monocyclic C3-C6 hydrocarbon, or a C8-C10 bicyclic hydrocarbon that is completely saturated or that contains one or more units of unsaturation, but which is not aromatic, that has a single point of attachment to the rest of the molecule, or a C9-C16 tricyclic hydrocarbon that is completely saturated or that contains one or more units of unsaturation, but which is not aromatic, that has a single point of attachment to the rest of the molecule.

As used herein, the term “alkyl” is given its ordinary meaning in the art and may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has 1-20 carbon atoms in its backbone (e.g., C1-C20 for straight chain, C2-C20 for branched chain), and alternatively, 1-10 carbon atoms, or 1 to 6 carbon atoms. In some embodiments, a cycloalkyl ring has from 3-10 carbon atoms in their ring structure where such rings are monocyclic or bicyclic, and alternatively 5, 6 or 7 carbons in the ring structure. In some embodiments, an alkyl group may be a lower alkyl group, wherein a lower alkyl group comprises 1-4 carbon atoms (e.g., C1-C4 for straight chain lower alkyls).

As used herein, the term “alkenyl” refers to an alkyl group, as defined herein, having one or more double bonds.

As used herein, the term “alkynyl” refers to an alkyl group, as defined herein, having one or more triple bonds.

As used herein, the term “azide” is given its ordinary meaning in the art and may include an alkyl group, as defined herein, having one or more azide functional groups.

The term “heteroalkyl” is given its ordinary meaning in the art and refers to alkyl groups as described herein in which one or more carbon atoms is replaced with a heteroatom (e.g., oxygen, nitrogen, sulfur, and the like). Examples of heteroalkyl groups include, but are not limited to, alkoxy, poly (ethylene glycol), alkyl-substituted amino, tetrahydrofuranyl, piperidinyl, morpholinyl, etc.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, binaphthyl, anthracyi and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone of as part of a larger moiety, e.g., “heteroaralkyl,” or “heteroaralkoxy,” refer to groups having 5 to 10 ring atoms (i.e., monocyclic or bicyclic), in some embodiments 5, 6, 9, or 10 ring atoms. In some embodiments, such rings have 6, 10, or 14π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quatemized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. In some embodiments, a heteroaryl is a heterobiaryl group, such as bipyridyl and the like. The terms “heteroaryl” and “heteroar-,” as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido [2,3-b]-1,4-oxazin-3 (4H)-one. A heteroaryl group may be monocyclic, bicyclic, tricyclic, tetracyclic, and/or otherwise polycyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5-to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen.

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be monocyclic, bicyclic, tricyclic, tetracyclic, and/or otherwise polycyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation but is not intended to include aryl or heteroaryl moieties, as herein defined.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring.

The term “unsaturated,” as used herein, means that a moiety has one or more units of unsaturation.

The term “halogen” means F, Cl, Br, or I; the term “halide” refers to a halogen radical or substituent, namely —F, —Cl, —Br, or —I.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

The term “spiro compound” refers to a chemical compound that presents a twisted structure of two or more rings, in which at least 2 rings are linked together by one common atom, e.g., a carbon atom. When the common atom is located in the center of the compound, the compound is referred to as a “spirocentric compound.” The common atom that connects the two or more rings is referred to as the “spiro-atom.” When such common atom is a carbon atom, it is referred to as the “spiro-carbon.”

Unless otherwise stated, all tautomeric forms of the compounds of the invention are within the scope of the invention.

Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a 11C- or 13C- or 14C-enriched carbon are within the scope of this invention.

Reference will now be made in detail to exemplary embodiments of the disclosed technology, examples of which are illustrated in the accompanying drawings and disclosed herein. Wherever convenient, the same references numbers will be used throughout the drawings to refer to the same or like parts.

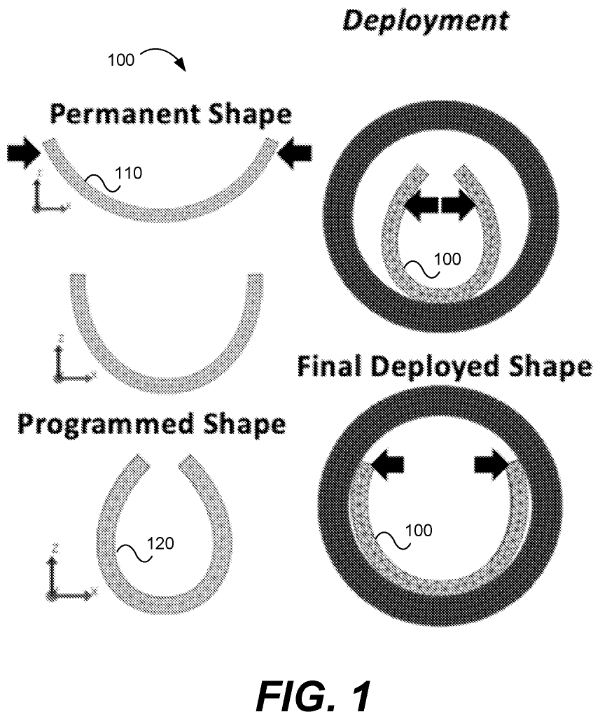

FIG. 1 illustrates an implant material 100 having a first shape 110 and a second shape 120. The implant material 100 can comprise a shape memory polymer, and the shape memory polymer can confer properties of the first shape 110 and the second shape 120 to the implant material 100. The shape memory polymer can comprise at least one monomer unit of glycerol and at least one monomer unit of dodecanedioate. Additional other monomer units can be present in the shape memory polymer as desired. Additives can also be added to the shape memory polymer, such as porogens, surfactants, binders, emulsifiers, and the like.

The ratio of the glycerol to the dodecandeioate can be altered as desired to confer certain properties to the implant material 100. For example, the molar ratio of the at least one monomer unit of glycerol to the at least one monomer unit of dodecanedioate can be from approximately 10:1 to approximately 1:10 (e.g., from 9:1 to 1:10, from 8:1 to 1:10, from 7:1 to 1:10, from 6:1 to 1:10, from 5:1 to 1:10, from 4:1 to 1:10, from 3:1 to 1:10, from 2:1 to 1:10, from 1:1 to 1:10, from 10:1 to 1:9, from 10:1 to 1:8, from 10:1 to 1:7, from 10:1 to 1:6, from 10:1 to 1:5, from 10:1 to 1:4, from 10:1 to 1:3, from 10:1 to 1:2, from 10:1 to 1:1, or from 5:1 to 1:5).

The shape memory polymer can also have a functionalized surface. The functionalized surface can comprise a plurality of suture holes laser cut into the functionalized surface. The suture holes, or other surface modulations, can be implemented in the implant device 100 to alter the mechanical properties of the implant device 100 as desired. The functionalized surface can comprise, for example, suture holes, grooves, wells, ribs, raised portions, other patterns, and the like. The patterns in the functionalized surface can be patterned using subtractive manufacturing. The functionalized surface can also comprise at least one functional group bonded to the shape memory polymer. The functionalized surface can be conjugated to improve the biocompatability of the implant material 100. For example, the at least one functional group can be a bioactive agent. The functionalized surface can also be altered to include a plurality of living and/or nonliving cells.

The shape memory polymer can take the first shape 110 at a first environmental temperature and the second shape 120 at a second environmental temperature. The shape memory polymer can have a melt transition temperature from approximately 25° C. to approximately 45° C. (e.g., from 26°° C. to 44° C., from 27° C. to 43° C., from 28° C. to 42° C., from 29°° C. to 41° C., from 30° C. to 40° C., from 31° C. to 39° C., from 32° C. to 38° C., from 33° C. to 37° C. from 34° C. to 36° C., from 30°° C. to 39° C., from 31° C. to 38° C., from 31° C. to 37° C., from 31°° C. to 36° C., or from 31° C. to 35° C.). The melt transition temperature can be greater than or equal to the first environmental temperature and less than the second environmental temperature. Alternatively, or in addition, the shape memory polymer can be an elastomer above the melt transition temperature and a thermoplastic in the below the melt transition temperature.

The first shape 110 can be considered a “permanent” or recovered shape. In other words, the implant material 100 can be configured to return to the first shape when no stimulus is present. The first shape 110 can be a curved or tubular shape. The second shape 120 can be a “programmed” or stimulated shape. In other words, the implant material 100 can be configured to take the second shape 120 in response to a stimulus, such as temperature, light, pH, and the like. For example, the implant material 100 can take the second shape in response to a temperature stimulus of the second environmental temperature being greater than the first environmental temperature. The second shape 120 can be a tubular shape comprising a cut along a longitudinal axis of the implant material 100.

By way of another example, the permanent shape can be a curve and the programmed shape can be tubular. When subjected to stimulus of a cold temperature, the implant material 100 can take the programmed shape and behave as a thermoplastic below the melt transition temperature. When implanted into a warm environment, the implant material 100 can recover to the permanent shape when implanted and behave as an elastomer above the melt transition temperature.

Furthermore, the implant material can have a biodegradation time when implanted in vivo from approximately 4 months to approximately 24 months (e.g., from 5 months to 23 months, from 6 months to 22 months, from 7 months to 21 months, from 8 months to 21 months, from 9 months to 20 months, from 10 months to 20 months, from 10 months to 15 months, from 5 months to 15 months, from 4 months to 10 months, or from 12 months to 24 months).

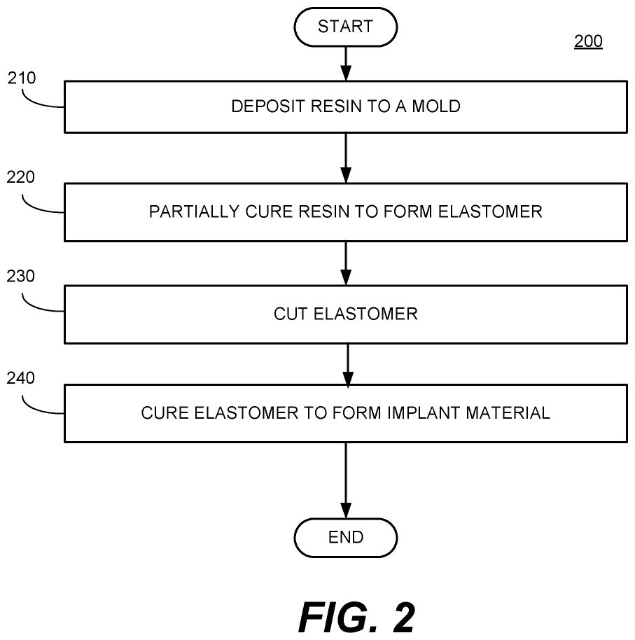

FIG. 2 is a flowchart of a method 200 of making an implant material 100. As shown in block 210, the shape memory polymer can be deposited in resin form into a mold. The mold can be 3D printed, and the mold can have various patterns to impart surface geometry to the functionalized surface of the implant material 100. The various patterns in the mold can have features having a size of approximately 5 microns or greater. The mold can also be surface treated with a nonfouling release agent, such as parylene. The method 200 can then proceed on to block 220.

In block 220, the shape memory polymer can be partially cured to move from the resin form to an elastomer form in the mold. The shape memory elastomer can also be released from the mold. As would be appreciated, the surface treatment of the mold can aid in releasing the shape memory elastomer from the mold. The method 200 can then proceed on to block 230.

In block 230, the partially cured shape memory elastomer can be cut. For example, the shape memory elastomer can be laser cut into a variety of shapes as desired, such as a patterned mesh. The cut shape memory elastomer can further have its surface geometry altered by subtractive manufacturing to impart additional patterns to the shape memory polymer. The various patterns added by subtractive manufacturing can have features having a size of approximately 5 microns or greater. The method 200 can then proceed on to block 240.

In block 240, the shape memory elastomer can be fully cured to form the implant material 100. The implant material 100 can be fully cured at a variety of temperatures, temporal durations, and pressures as desired to yield desirable properties in the implant material 100, such as crosslink density and toughness. The shape memory elastomer can also be fully cured in a permanent shape such that the implant material 100 takes on a permanent shape. For example, the shape memory elastomer can be fully cured on a roller such that the implant material 100 is a tubular shape. The method 200 can terminate after block 240. However, the method 200 can also proceed on to other method steps not shown.

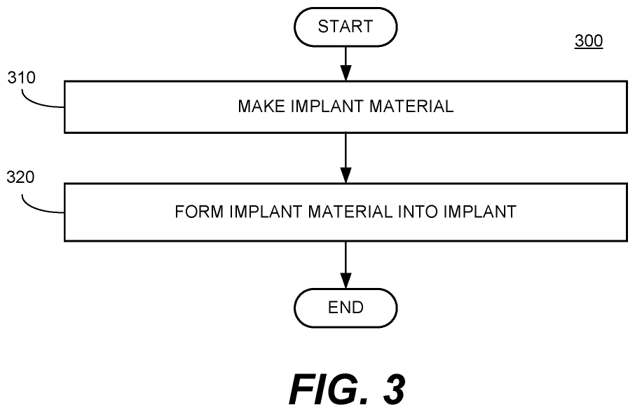

FIG. 3 is a flowchart of another method 300 of making an implant material 100. In block 310, the implant material 100 can be made through any of the methodologies as described herein. In block 320, the implant material 100 can be formed into an implant. The implant can be in the form of a sheet, a membrane, a mesh, a sponge, a patch, a molded medical device, or combinations thereof. The implant can also be functionalized as described herein such that the implant has a functionalized surface.

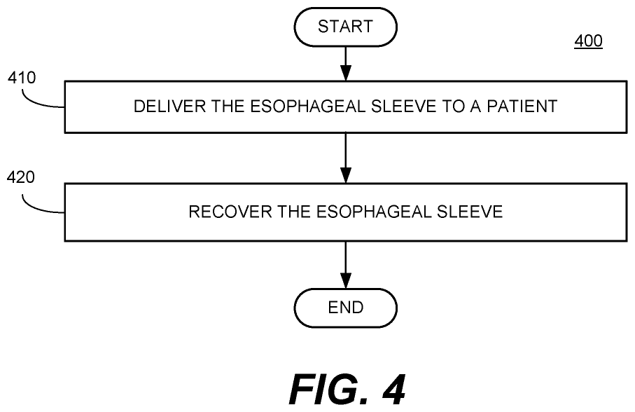

FIG. 4 is a flowchart of a method 400 of implanting an esophageal sleeve. As shown in block 410, the esophageal sleeve can be delivered to a patient. The implant material 100 can have a permanent shape of a curve and the programmed shape can be tubular. When subjected to stimulus of a cold temperature, the implant material 100 can take the programmed shape and behave as a thermoplastic below the melt transition temperature during the delivery of block 410. In block 420, the esophageal sleeve can recover to take the permanent shape when implanted. When implanted into a warm environment, the implant material 100 can recover to the permanent shape when implanted and behave as an elastomer above the melt transition temperature during the recovery in block 420.

Certain embodiments and implementations of the disclosed technology are described above with reference to block and flow diagrams of systems and methods and/or computer program products according to example embodiments or implementations of the disclosed technology. It will be understood that one or more blocks of the block diagrams and flow diagrams, and combinations of blocks in the block diagrams and flow diagrams, respectively, can be implemented by computer-executable program instructions. Likewise, some blocks of the block diagrams and flow diagrams may not necessarily need to be performed in the order presented, may be repeated, or may not necessarily need to be performed at all, according to some embodiments or implementations of the disclosed technology.

### EXAMPLES

The following examples are provided by way of illustration but not by way of limitation.

Esophageal anatomy in the esophageal atresia model can be idealized as concentric cylinders where the inner cylinder represents the mucosal layer, and the outer cylinder represents the smooth muscle layer. The esophageal segment can be assumed to be 100 mm long, and the inner esophageal lumen can be assumed to be 10 mm in diameter, the mucosal layer 1.2 mm thick, and the smooth muscle 2.3 mm thick using measurements. A 5,760-element finite element mesh comprised of 8-node hexahedral elements and 7872 nodes can be used for the esophageal models. Both the mucosa and smooth muscle tissue can be modeled as an anisotropic nonlinear elastic material capable of undergoing large deformation. For initial models, the strain energy function can be used:

$$W = {a}_{1} ( {I}_{1} - 3 ) + \frac{{a}_{2}}{{a}_{3}} [ {e}^{{{a}_{3} ( {I}_{4} - 1 )}^{2}} - 1 ] \qquad eq . 1$$

$${I}_{1} = {λ}_{1}^{2} + {λ}_{2}^{2} + {λ}_{3}^{2}$$

$${I}_{4} = {N}_{i} {C}_{ij} {N}_{j}$$

where W denotes the strain energy function, λᵢ denote stretch ratios in the 1(x), 2(y) and 3(z) directions, Nᵢ are unit normal vectors in the undeformed configuration representing principal fiber directions in the tissue, Cᵢⱼ is the right Cauchy deformation tensor, I₁ is the first invariant of Cᵢⱼ, I₄ is a pseudo-invariant of Cᵢⱼ defined in terms of stretch ratios, and a₁, a₂, and a₃ are coefficients of the constitutive model. It can be assumed that N₃ aligns along the long axis z of the esophagus and that the N₁ and N₂ align at an angle ϕ from the x axis. From, a₁=0.00015 MPa, a₂=0.0003 MPa, a₃=1.17 and ϕ=48.2° for the mucosa layer and a₁=0.00017 MPa, a₂=0.00137 MPa, a₃=4.62 and ϕ=55.3° for the smooth muscle layer.

Results show all body force magnitudes can fall within experimental data especially at higher gap displacements above 30 mm. Force magnitudes greater than 0.75 N can be associated with excessive scarring and disruption of the anastomosis, indicating that 0.75 N can be a safe force limit which appears to occur at gap displacements above 3.0 to 3.5 cm.

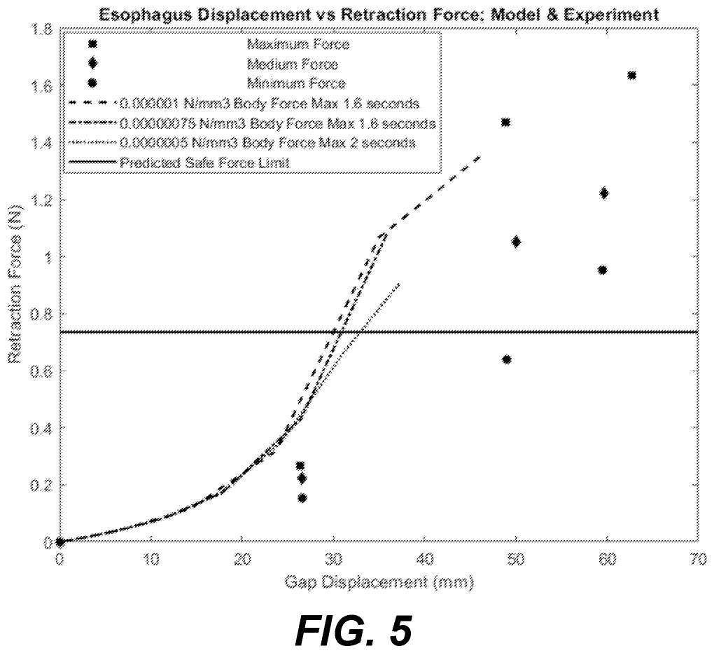

Since the esophagus ends can be approximated with incorporated sutures to simulate anastomosis, a method can be implemented to model the inherent force to approximate the ends which can subsequently pull apart the esophagus ends if they are not tied together. This retraction force can be represented as a body force acting over 2 seconds that linearly increased closer to the anastomosis. A model can be created with the esophagus model in contact with a rigid plate to determine the retraction force (FIG. 5). Body forces of 5e⁻⁷, 7.5e⁻⁷, and 1e⁻⁷ N/mm³ can be applied over 2.5 seconds with the resultant total retraction force determined by contact between the esophageal segment with the plate. In this scenario, the effect of a larger gap can be modeled resulting from a larger retraction force and can be simulated by increasing the time over which the body forces are applied. For example, a 1.5 second body force application (1500 ms) can result in a 20 mm gap while a 2.2 second (2200 ms) body force application can result in a 40 mm gap. Since the body forces can be time dependent, a nonlinear structural dynamics analysis can be performed for all cases, using consistent units of g/mm³ for density to calculate mass, N for force, MPa for constitutive properties, and mm for length. This reaction force versus gap displacement can be compared to experimental results for esophagus force as shown in FIG. 5. Symmetry can be enforced by fixing the middle nodes of the esophagus against lateral motion.

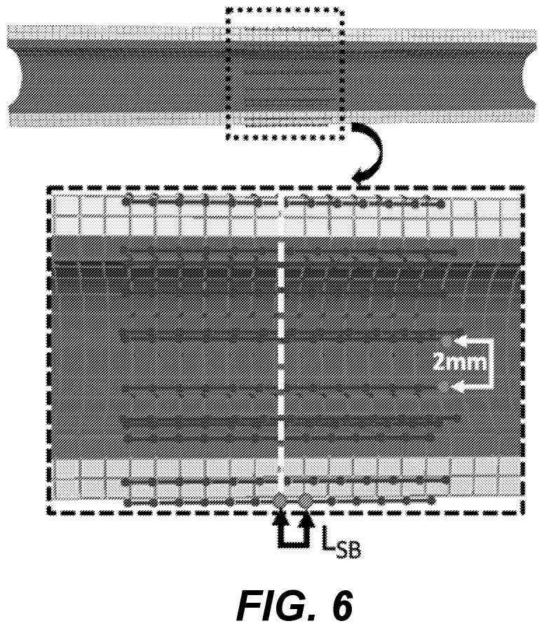

To simulate anastomosis of an esophageal atresia segment, the distinct segments of the esophagus can be approximated end to end, with a nonlinear sliding elastic contact allowed between the ends with a friction coefficient of 0.1. The esophagus as noted can be modeled as a nonlinear elastic material using 5,760 8-node hexahedral elements and 7872 nodes in the nonlinear finite element code FEBio version 3.5. Sutures can be nonlinear as they carry tension but not compression. The sutures can therefore be modeled as nonlinear springs that generate a specific force under tensile displacement, but zero force under compression. To calibrate the spring load curves, a solid model of suture 250 microns in diameter, 2 mm long can be stretched between rigid plates and modeled as a linear elastic material with Young's modulus of 745 MPa. The nonlinear spring tensile force displacement curve can then be calibrated to the reaction force from the solid model results. For suture modeling, springs can be placed at the outer and inner esophagus wall circumferentially every 2 mm (FIG. 6 shows a split view of the model with spring sutures shown in dark blue) connecting nodes from one side to the other side of the anastomosis. Different suture bites (e.g., the distance from the anastomosis site to the end of the suture away from the anastomosis) of 1.25 mm, 2.5 mm, 3.75 mm, and 5 mm can be used.

The effect of suturing the esophagus into a biomaterial sleeve to support the esophagus against the retraction forces can also be modeled. The biomaterial sleeve design with a 17 mm inner diameter, 2 mm wall thickness, and 4 longitudinal by 9 circumferential 2 mm suture holes can be generated using a MATLAB program and converted into an STL file. This STL file can be meshed using 65,569 10-node tetrahedral elements (124,353 nodes) in FEBio studio version 1.6. The sleeve can be modeled as a nonlinear elastic bioresorbable elastomer poly (glycerol dodecanedioate)—PGD using a 1-term Ogden model:

$$W = \frac{{a}_{1}}{{a}_{2}^{2}} ( {λ}_{1}^{{a}_{2}} + {λ}_{2}^{{a}_{2}} + {λ}_{3}^{{a}_{2}} - 3 ) \qquad ( eq . 2 )$$

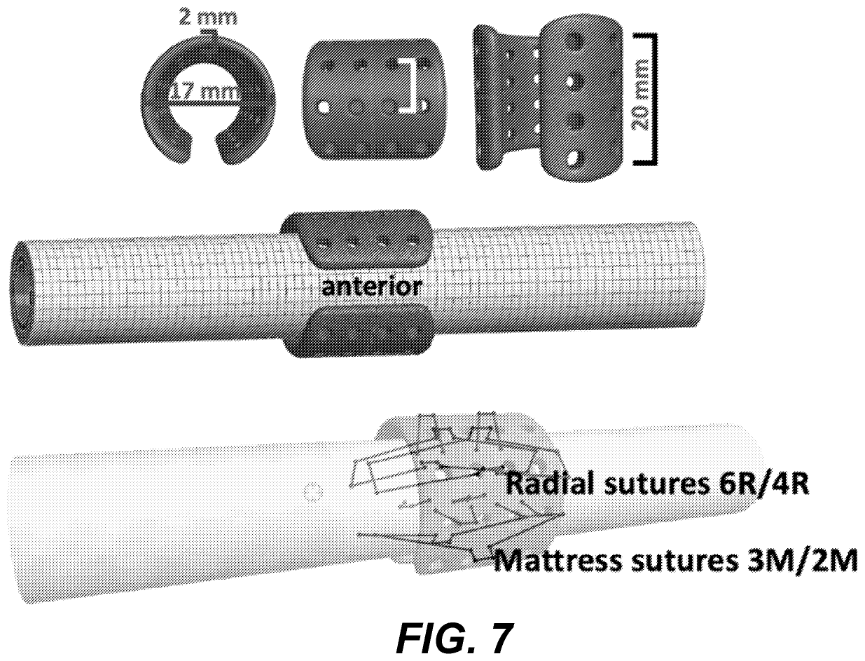

with a₁=4.4 MPa and a₂=0.096 and the stretch ratios as defined in eq. 1. Two base suture patterns can be used to connect the sleeve to the esophagus, consisting of both mattress and radial sutures. In the first mattress suture circuit, the suture can be first connected on one side of the sleeve opening from the mucosa smooth muscle junction to the inferior sleeve suture hole closest to the anastomosis, across to the adjacent superior sleeve suture hole back to the mucosa smooth muscle junction at the opposite side of the anastomosis. Then, the suture can be continued circumferentially across the sleeve opening at the mucosa smooth muscle junction, back to the first inferior suture hole at the anastomosis across to the first superior sleeve suture hole back to the sleeve edge and across to the originating point of the suture (FIG. 7). A second mattress suture circuit can be completed on the opposite side of the esophagus from the first (FIG. 7). Two radial suture loops between the sleeve and the mucosa smooth muscle junction on both the inferior and superior sides of the anastomosis can be placed at each edge of each mattress suture circuit for a total of eight radial sutures (FIG. 7). The second base suture pattern can comprise three mattress suture circuits located at the sleeve opening and at 120° and 240° from the suture opening. Four radial sutures can be located on each side of each mattress suture circuit for a total of twelve radial sutures (FIG. 7). Perturbations for each base suture configuration can be created by eliminating all the radial sutures leaving mattress sutures only or eliminating all the mattress sutures leaving radial sutures only.

To investigate the impact of anastomotic sleeve properties, the material parameters for the sleeves can be changed from the nonlinear elastic properties of the sleeve. A low cure formulation (PGD_{L}) can be selected to determine the impact of polymer crosslink density on reducing anastomotic strain. PGD_{L} can be modeled as a one term Ogden material a₁=2.5 MPa and a₂=0.06. Additionally, poly(lactide-co-caprolactone) (PLCL) and polycaprolactone (PCL) can be modeled as linear isotropic elastic materials. PLCL can have an elastic modulus of 148 and a Poisson's ration of 0.3 while PCL can be modeled with an elastic modulus of 298 and a Poisson's ratio of 0.3.

Results can be characterized using displacement magnitude U, effective LaGrangian strain E, and strain energy density calculated in FEBio and defined below:

$$Displacement Magnitude : U = \sqrt{{u}_{1}^{2} + {u}_{2}^{2} + {u}_{3}^{2}}$$

$$Effective LaGrangian Strain : E = \sqrt{\frac{1}{2} [ {( {E}_{11} - {E}_{22} )}^{2} + {( {E}_{11} - {E}_{33} )}^{2} + {( {E}_{22} - {E}_{33} )}^{2} ] + 3 ( {E}_{12}^{2} + {E}_{13}^{2} + {E}_{23}^{2} )}$$

where Eᵢⱼ is the finite Lagrange strain tensor:

$${E}_{ij} = \frac{1}{2} ( \frac{\partial {u}_{i}}{\partial {X}_{j}} + \frac{\partial {u}_{j}}{\partial {X}_{i}} + \frac{\partial {u}_{k}}{\partial {X}_{i}} \frac{\partial {u}_{k}}{\partial {X}_{j}} )$$

Strain Energy Density: Strain energy function W defined for the Gasser-Ogden-Holzapfel model in eq. 1. These results can be calculated within six layers, with each layer 1.25 mm in length. Starting at the anastomosis site the six layers extend 7.25 mm from the anastomosis site both inferiorly and superiorly.

Total reaction force versus gap displacement for each body approximation force level can be compared against experimental force vs. gap displacement measurements made in vivo on pig esophagus. All body force levels can fall within the scatter reported in FIG. 5. Since the lowest body force application of 5.e⁻⁷ N/mm3 fell closest to the 2.7 cm gap approximation force, this body force can be used for all subsequent analyses.

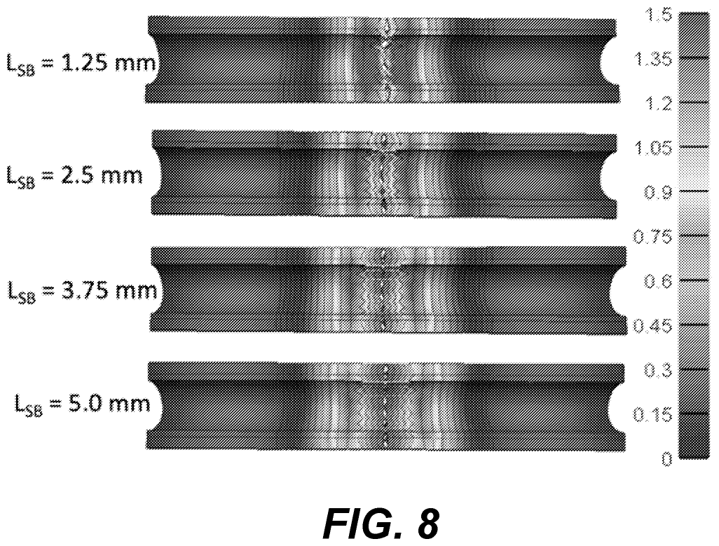

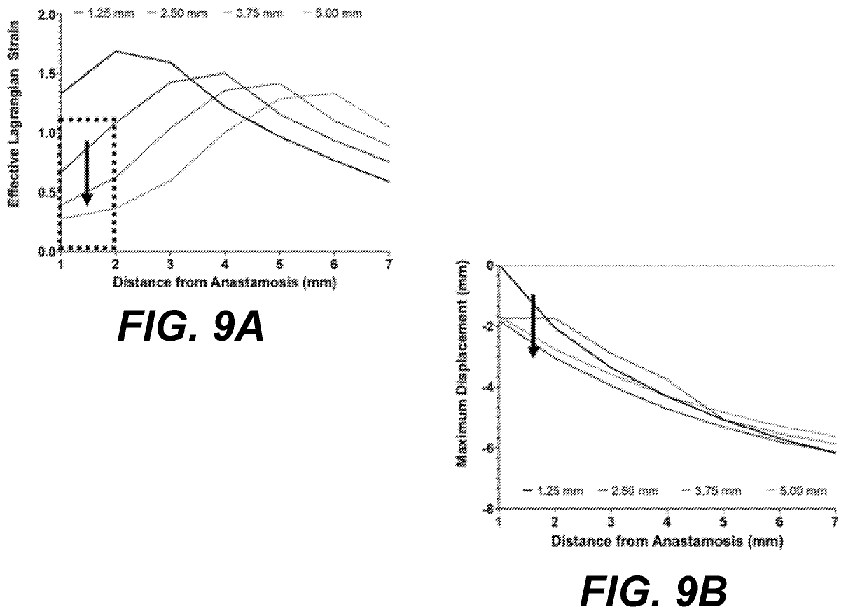

Anastomosis with different suture “bite” lengths (L_{SB}), length of the suture in the esophagus wall from the anastomosis site, can reveal that longer suture bites can reduce the total effective LaGrangian strain, displacement magnitude, and strain energy density in the anastomosis zone (FIG. 6, black arrows). Sutures can be placed 2 mm apart circumferentially along the esophageal lumen (FIG. 6). Effective LaGrangian strain at the anastomosis site can decrease with increasing suture length (FIG. 8, FIG. 9A). Difference in LaGrangian strain across suture bite lengths can be greatest within the first 2 mm of the anastomosis site (FIG. 9A). Longer suture bite lengths can decrease the displacement magnitudes (FIG. 9B). Increasing suture bite length can also result in a decrease in effective strain distribution near the anastomosis, pushing areas of high strain away from the anastomosis region to the suture end. There can be a decrease in maximum displacement.

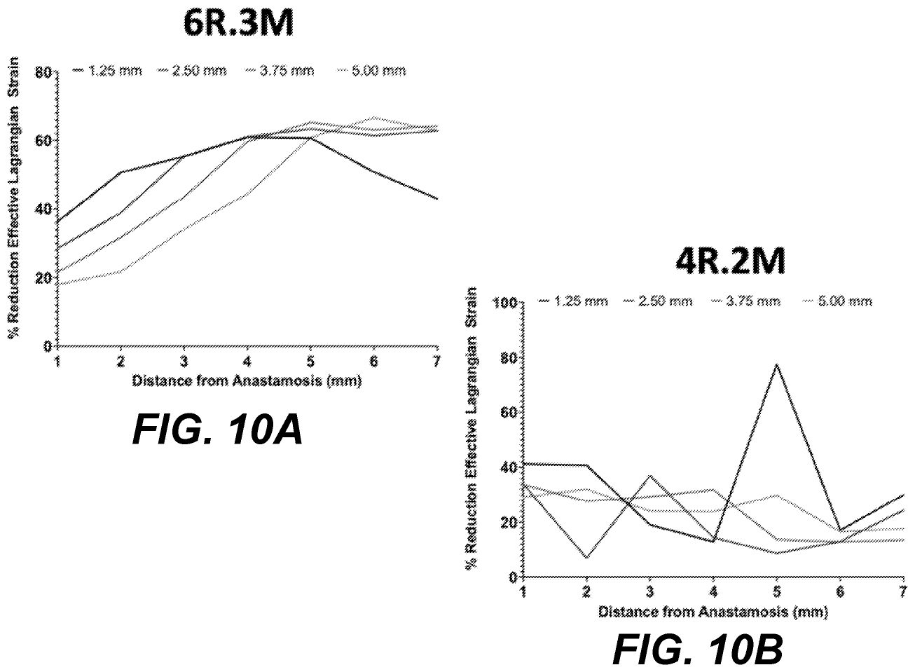

Each suture bite can exhibit a reduction in high strain regions within the anastomosis zone with the presence of the sleeve. The rationale for using both mattress and radial sutures can be that the mattress sutures can better resist longitudinal deformation, while the radial suture can better resist radial deformation. Results for both sleeve suture configurations (6R.3M and 4R.2M) can demonstrate a substantial reduction (most cases 50% or more at the anastomosis site) in anastomosis strain levels with the sleeve compared with no sleeve (FIGS. 10A and 10B). There can be a greater reduction in displacement magnitude for the 4R.2M suture configuration compared to the 6R.3M suture configuration near the anastomosis. There can be an equivalent reduction in strain energy density at the anastomosis between suture bite patterns.

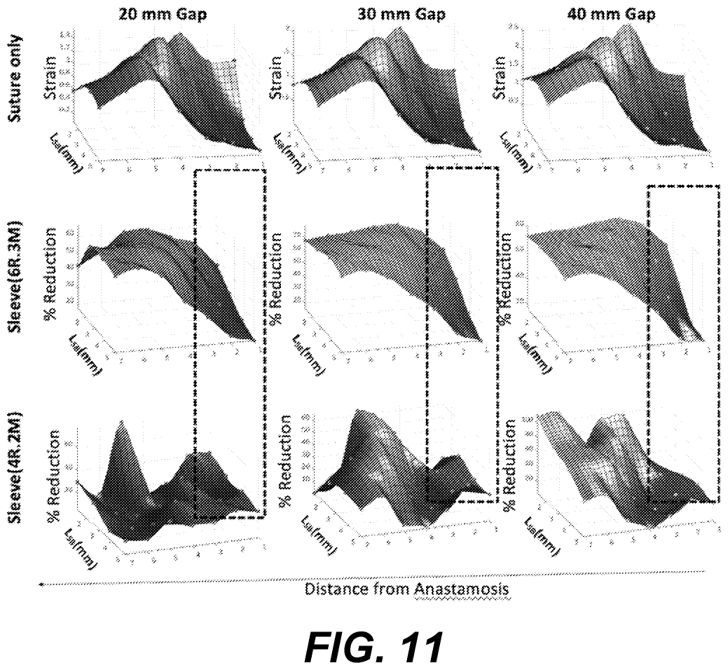

Longer gap atresias can exhibit higher retraction forces at the anastomosis and consequently increased radial and axial tension from primary repair. There can be a greater LaGrangian strain for longer gap atresias (30 and 40 mm) than the smaller gap atresias (FIG. 11). The effective strain reduction % at the anastomosis using a sleeve with 6R.3M compared to 4R.2M suture pattern can become more pronounced at higher esophageal gap lengths. Interestingly, there can be greater reductions in strain 5-7 mm away from the anastomosis compared to 1-4 mm away from the anastomosis when using the sleeve. Without wishing to be bound by any particular scientific theory, this can be explained by the suture pattern used to affix the sleeve. The radial sutures used to affix the sleeve can reduce longitudinal tension whereas the mattress sutures can reduce radial tension. There can be a greater reduction in strain where these forces converge. Consequently, the 20 mm, 30 mm, and 40 mm gap models can exhibit maximum reductions in strain at slightly different distances from the anastamosis site. Sleeve repair models with fewer sutures can have a greater maximum reduction in strain.

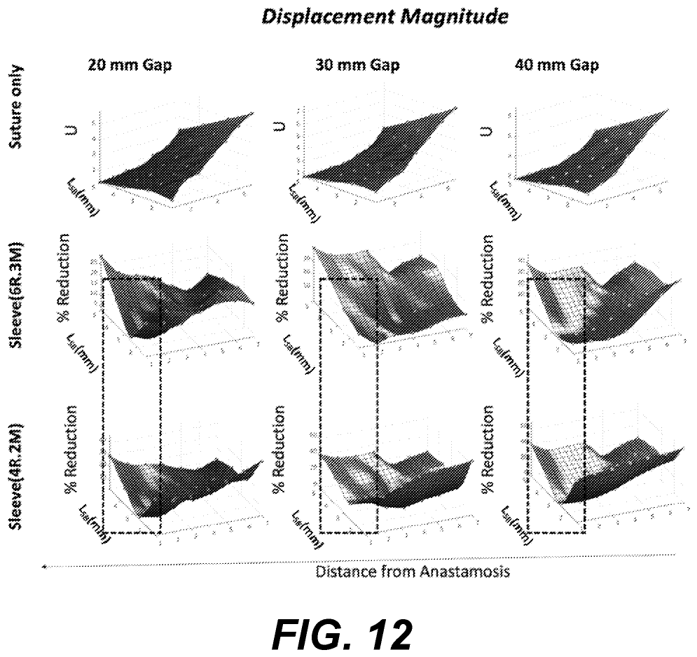

Similarly, the displacement magnitude of the anastomosis can be greater for long gap atresias compared to short gap atresias. The greatest reduction in esophageal displacement magnitude can occur at the anastamosis using a 4R.2M suture pattern and a 2.5-3.75 mm radial suture bite across all esophageal gap lengths (FIG. 12). In both 6R.3M and 4R.2M suture methods, the overall % reduction can decrease to a lesser extent 4 mm away from the anastomosis site.

Without wishing to be bound by any particular scientific theory, the resistance to radial extension provided by the radial sutures and interaction with mattress sutures can drive this reduction difference between regions proximal vs. distal from the anastomosis site. Additionally, although the deformation of the tissue resulting from suture perforation was not modeled, there can be a preference for 4R.2M approach due to consideration regarding the structural integrity of the tissue.

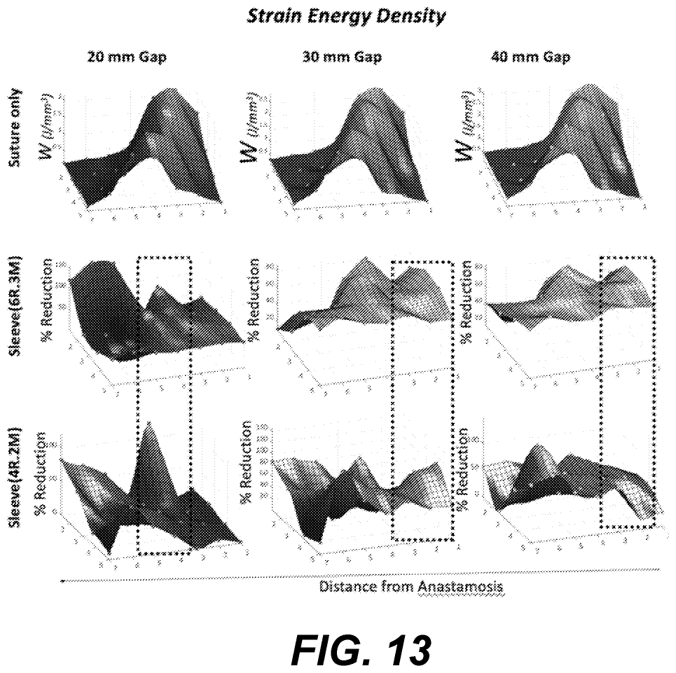

Strain energy density at the anastomosis can increase with increasing gap length (FIG. 13). Similarly, applying a sleeve for repair regardless of suture pattern can result in greater reduction in strain energy density proximal to the anastomosis compared to primary repair. There can be a negligible difference in strain energy density at the anastomosis between different suture bite patterns.

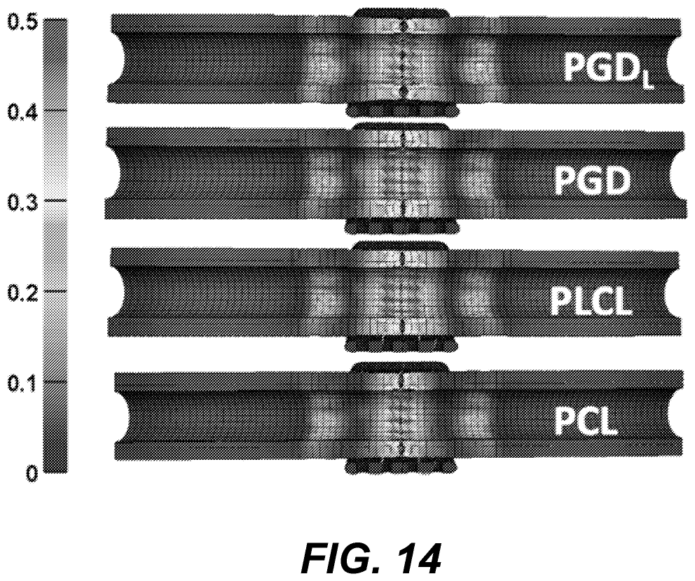

Changing the material properties of the sleeve can impact strain at anastomosis (FIG. 14). Materials with greater stiffness (PGD_{L}<PGD<PLCL<PCL) can exhibit lower strain at the anastomosis with a 20 mm gap length. At higher gap lengths, there can be a greater reduction in strain energy for all sleeves (FIG. 5). However, this reduction in strain at 30 mm gap length can be greater using a PLCL sleeve compared to the other materials, which can indicate sleeves with greater stiffness. Similarly, PLCL can exhibit a greater reduction in strain energy density and displacement magnitude at the anastomosis compared to the other materials. Although PGD can be a nonlinear elastomer more ideally suited for the repair of esophageal tissues, there can be a tradeoff between stiffness and elasticity of the material. With further material development using composite matrices, it can be possible to tune the nonlinear material properties to meet the requirements for various gap defects.

When a sleeve is used with a 4R.2M suture pattern, there can be significantly greater reductions in strain energy, displacement magnitude, and strain energy density for PLCL as previously noted. However, PGD_{L} and PLCL can exhibit lower anastomotic strains and displacement with respect to suturing compared to PGD and PCL, respectively. Both strain energy density and displacement magnitude can be reduced to a greater extent by the PLCL sleeve compared to the PGDL sleeve when a 4R.2M suture pattern was used. However, there can be a greater reduction in strain energy using a PGDL sleeve. Taken together this suggests a discernable interaction between suture methods, linear and nonlinear material properties, and gap length.

Disclosed herein is a framework for evaluating esophageal atresia repair, allowing for the computational testing of both clinical and device engineering hypotheses impacting repair. As expected, longer gap atresias, simulated by higher retraction body forces, can result in greater anastomotic strain, displacement magnitudes, and strain energy. Although increasing suture bite length can alleviate the anastomotic strain to a degree, the longer gap atresia can require a device to reduce longitudinal and radial tension at the anastomosis. In general, presence of a sleeve can reduce anastomotic strain, displacement magnitude, and strain energy density compared to suturing alone. Varying number of radial and mattress sutures in conjunction with the sleeves, can reveal that the 4R.2M suture pattern can reduce anastomotic tension as much or more than the 6R.3M suture pattern indicating that more suturing within the sleeve is not necessarily better.

Increasing stiffness of the sleeve did not correlate with reduced strain. An interaction between suture method and sleeve material can be revealed. Although passive esophageal properties can be considered for this study, an active esophageal model can consider forces imparted on the sleeve from smooth muscle contractility. Softer materials, especially those suited for the nonlinear mechanics of esophageal tissues, can be more likely to accommodate axial and radial forces below levels that may lead to complications. Increasing material stiffness can reduce anastomotic tension, but materials of greater stiffness tend to have longer degradation times, potentially complicating healing.

There can be a complex interaction between sleeve stiffness and suturing pattern such that sleeve stiffness and the number of sutures can require optimization for reducing anastomotic tension. These complex interactions can be more readily testable and evaluated using computational and numerical methods that can generate focused hypotheses to be tested experimentally. In addition to initial stiffness, the material properties as tissue grows and device degrades can also be considered for design. Understanding the degradation rate and designing for growth can support and guide anostamotic healing. The disclosed sleeves can also be used as a vehicle to locally administer growth factors, anti-inflammatory drugs, or angiogenic factors to promote healing and improve repair outcomes. Another advantage of the disclosed biomaterial sleeve use can be the ability to use the material as a vehicle to improve healing. Delivery of bioactive molecules can be possible with a sleeve, thereby potentially augmenting the effect of the sleeve in improving EA repair by reducing anastomotic tension and guiding healing.

To accommodate initial set up of the anastomosis including sutures, body forces can be used to implement the inherent tensile stresses imparted due to stretching the esophagus. The body force magnitude and time duration chosen can produce results consistent with in vivo experimental data in pigs. Finally, the use of springs to mimic suturing can be an assumption. Suturing can likely support tensile forces but not compression, but the current implementation of nonlinear spring behavior in FEBio does not allow pre-tensioning, which may occur during surgery.

Disclosed herein is a computational framework for approximating esophageal anastomotic tension to study the impact of primary and device supported repair using a polymeric sleeve into the esophagus is sutured. The esophagus can be modeled as an idealized concentric cylinder comprised of mucosal and muscle layers described by nonlinear strain energy functions incorporating a mixed fiber model with a Neo-Hookean base material (FEBIO studio). Sutures can be modeled as nonlinear elastic springs carrying only tension, and shape memory biodegradable elastomeric polymeric sleeves comprised of poly(glycerol dodecanedioate) can be modeled as nonlinear elastic materials using one term Ogden parameters. Elastomeric sleeves can be compared to isotropic elastic thermoplastic polymers, polycaprolactone and poly(L-lactide co-caprolactone), as used in esophageal repair. The impact of suture bite (length of suture from anastomosis), sleeve material properties, sleeve suture strategy, and gap length can be evaluated with respect to anastomotic LaGrangian strain, displacement magnitude, and strain energy density.

With increasing gap length, there can be an increase in anastomotic strain, displacement magnitude, and strain energy density. Increasing the suture bite length can decrease strain at the anastomosis. Application of the sleeve can reduce strain, displacement, and strain energy to a greater extent in longer gap atresia. Increasing the number of sutures to apply the sleeve did not decrease the esophageal strain compared to sleeves with a lesser number of sutures. Sleeve material testing can reveal an interplay between the nonlinear mechanical properties of the selected materials and their contribution to reducing anastomotic tension.

Taken together, the present disclosure can provide a framework for computational verification of design broadly addressing clinical procedure optimization, material design, and device design for surgical repair of esophageal atresia.

While the present disclosure has been described in connection with a plurality of exemplary aspects, as illustrated in the various figures and discussed above, it is understood that other similar aspects can be used, or modifications and additions can be made to the described aspects for performing the same function of the present disclosure without deviating therefrom. For example, in various aspects of the disclosure, methods and compositions were described according to aspects of the presently disclosed subject matter. However, other equivalent methods or composition to these described aspects are also contemplated by the teachings herein. Therefore, the present disclosure should not be limited to any single aspect, but rather construed in breadth and scope in accordance with the appended claims.

1. A device comprising:
a bioresorbable scaffold comprising:
a shape memory polymer comprising:
at least one monomer unit of glycerol; and
at least one monomer unit of dodecanedioate; and
a functionalized surface.

2. The device of claim 1, wherein;
the device is an esophageal sleeve device;
the bioresorbable scaffold takes a first shape at a first environmental temperature;
the bioresorbable scaffold takes a second shape at a second environmental temperature;
the second environmental temperature is greater than the first environmental temperature;
the functionalized surface is modified to have a biology corresponding to a patient; and
the esophageal sleeve device is configured to be implanted in the patient in the first shape at the first environmental temperature.

3. The device of claim 1, wherein the shape memory polymer has a melt transition temperature from approximately 25° C. to approximately 45° C.

4. The device of claim 1, wherein the shape memory polymer has a melt transition temperature greater than or equal to the first environmental temperature and less than the second environmental temperature.

5. The device of claim 1, wherein the shape memory polymer has a melt transition temperature;
wherein the shape memory polymer is an elastomer above the melt transition temperature; and
wherein the shape memory polymer is a thermoplastic below the melt transition temperature.

6. An esophageal sleeve device, comprising:
a bioresorbable scaffold comprising:
a shape memory polymer comprising:
at least one monomer unit of glycerol; and
at least one monomer unit of dodecanedioate; and
a functionalized surface modified to have a biology corresponding to a patient;
wherein the bioresorbable scaffold has a first shape, a second shape, and a melt transition temperature;
wherein the bioresorbable scaffold takes the first shape at a first environmental temperature and the second shape at a second environmental temperature;
wherein the second environmental temperature is greater than the first environmental temperature;
wherein the melt transition temperature is greater than or equal to the first environmental temperature and less than the second environmental temperature; and
wherein the first shape is different from the second shape.

7. The esophageal sleeve device of claim 6, wherein the first shape is a curved or tubular shape; and
wherein the second shape is a tubular shape.

8. The esophageal sleeve device of claim 6, wherein the functionalized surface comprises a plurality of suture holes.

9. The esophageal sleeve device of claim 6, wherein the functionalized surface comprises at least one functional group.

10. The esophageal sleeve device of claim 9, wherein at least one functional group comprises a bioactive agent.

11. The device of claim 1, wherein the molar ratio of the at least one monomer unit of glycerol to the at least one monomer unit of dodecanedioate is from approximately 10:1 to approximately 1:10.

12. The esophageal sleeve device of claim 6, wherein the bioresorbable scaffold has a biodegradation time when implanted in vivo from approximately 2 months to approximately 24 months.

13. A method of implanting an esophageal sleeve device comprising:
implanting the device of claim 1 in a patient, wherein the bioresorbable scaffold is in a permanent shape after the implanting.

14. The method of claim 13 further comprising:
delivering the device of claim 1, wherein the bioresorbable scaffold is in a programmed shape during the delivering and prior to the implanting;
wherein the bioresorbable scaffold takes the programmed shape at a first environmental temperature;
wherein the bioresorbable scaffold takes the permanent shape at a second environmental temperature;
wherein the second environmental temperature is greater than the first environmental temperature; and
wherein the functionalized surface is modified to have a biology corresponding to the patient.

15. (canceled)

16. The method of claim 14, wherein the shape memory polymer has a melt transition temperature from approximately 25° C. to approximately 45° C.; and
wherein the melt transition temperature is greater than or equal to the first environmental temperature and less than the second environmental temperature.

17. The method of claim 16, wherein the melt transition temperature is from approximately 31° C. to 35° C.

18. The method of claim 16, wherein the shape memory polymer is an elastomer above the melt transition temperature and a thermoplastic below the melt transition temperature.

19. The method of claim 14, wherein the programmed shape is a curved or tubular shape.

20. The method of claim 13, wherein the permanent shape is a tubular shape.

21. (canceled)

22. The method of claim 13, wherein the functionalized surface comprises at least one functional group.

23. The method of claim 22, wherein at least one functional group comprises a bioactive agent.

24. The method of claim 13, wherein the molar ratio of the at least one monomer unit of glycerol to the at least one monomer unit of dodecanedioate is from approximately 10:1 to approximately 1:10.

25. The method of claim 13, wherein the bioresorbable scaffold has a biodegradation time when implanted in vivo from approximately 2 months to approximately 24 months.
